(19) United States
(12) Patent Application Publication
(10) Pub. No.: US 2026/0345977 A1
(43) Pub. Date: Nov. 5, 2026

(54) CERVICAL INTRODUCER

(71) Applicant: Azmina Karukappadath, Baltimore, MD (US)

(72) Inventor: Azmina Karukappadath, Baltimore, MD (US)

(21) Appl. No.: 19/657,532

(22) Filed: Apr. 24, 2026

Related U.S. Application Data
(60) Provisional application No. 63/798,562, filed on May 2, 2025.

(51) Int. Cl. A61M 29/00; A61F 6/18

(52) U.S. Cl. CPC A61M 29/00; A61F 6/18; A61M 2205/02; A61M 2210/1433

(57) ABSTRACT

A novel trans-cervical uterine entry device or a cervical introducer which generally comprises a hollow threaded dilator body and cervical stoppage cone. The hollow threaded cervical dilator body may feature distal transverse slits to accommodate passage of instruments through the device. This cervical introducer allows gynecologic practitioners to access the uterine cavity from the vagina through a wide variety of cervical anatomies with minimal cervical trauma and pain to a patient.

### STATEMENT REGARDING FEDERALLY SPONSORED RESEARCH OR DEVELOPMENT

Not applicable.

### COPYRIGHT NOTICE

A portion of this disclosure contains material which is subject to copyright protection. The copyright owner has no objection to the photocopy reproduction by anyone of the patent document or the patent disclosure in exactly the form it appears in the Patent and Trademark Office patent file or records, but otherwise reserves all copyright rights whatsoever. 37 C.F. R 1.71(d).

### REFERENCE TO RELATED APPLICATIONS

This application claims the benefit of U.S. Provisional Application No. 63/798,562, filed May 2, 2025, entitled “CERVICAL INTRODUCER: A NOVEL ATRAUMATIC TRANSCERVICAL UTERINE ENTRY DEVICE.” The benefit under 35 USC § 119(e) of the United States provisional application is hereby claimed, and the aforementioned application is hereby incorporated herein by reference in its entirety.

### BACKGROUND OF THE INVENTIVE CONCEPT

### 1. Field of the Invention

The present inventive concept relates to a cervical introducer, and more specifically, to an atraumatic cervical introducer for gynecologic procedures, which provides a hollow self-advancing threaded cervical dilator body that enables passage of medical instruments through a cervical canal while preventing traumatic injury during such insertions.

### 2. Description of the Related Art

Several common OB/GYN procedures necessitate transcervical access to a uterus, including intrauterine device (IUD) placement, endometrial biopsy (EMB), and hysteroscopy. These procedures are typically performed in an outpatient setting without anesthesia and can be extremely painful for patients.

Transcervical uterine access is usually established through use of a tenaculum, a surgical instrument with sharp hooks, which punctures and clamps onto the cervix to provide stabilizing counter-traction while inserting instruments through the cervix. However, this method of entry often causes cervical trauma and significant pain to the patient, as well as bleeding and a higher risk of infection. Additionally, the tenaculum often fails to grant access through cervixes that are flush or stenosed, or those with high or narrow internal ora.

Various attempts have been made to improve upon or replace the traditional tenaculum, including devices with modified hooks, suction-based stabilizers, or atraumatic graspers. However, these alternatives often fail to provide adequate stabilization, are cumbersome to use, and are not compatible with non-standard cervical shapes as mentioned above.

U.S. Pat. No. 7,105,007 by Hibler is directed to a cervical medical device for dilating a cervical canal. This device includes an elongated member with an expandable anchoring component attached to a distal tip thereof. An expandable dilating member is attached to the elongated member for dilating the cervical canal. This device relies on inflatable/expandable components for anchoring and dilation of the cervical canal, and focuses on fluid/gas-based expansion and sealing for hysteroscopy. To insert and use this device, a provider will be required to also use a tenaculum.

U.S. Pat. No. 6,485,410 by Loy is directed to a hysteroscope port including a cylindrical cannula dimensioned for insertion through a vagina to penetrate a cervix. This device uses a balloon element having a non-smooth surface positioned adjacent a distal end of the cannula for dilating the cervix and for maintaining the cervix in a dilated state and relies on fluid-based dilation and friction via surface texture.

U.S. Pat. No. 10,660,670 by Niver is directed to a cervical dilator including a shaft and a balloon to guide the dilator through a cervix to seat the balloon in an uninflated state within the cervix. After the balloon is seated within the cervical canal a predetermined amount of fluid is introduced into the balloon cavity to fill the balloon to an inflated state, at which point a waiting period is required to allow cervical dilation. Then a tension must be applied to the exterior of the shaft to remove the shaft and the balloon from the cervix. This device will also necessitate use of a tenaculum.

Accordingly, there remains a need for a device that can achieve trans-cervical uterine access without puncturing tissue to minimize patient discomfort, reduce the risk of complications, and enhance procedural outcomes for all cervical anatomies.

There is also a need for a device that can achieve trans-cervical uterine access without the use of a balloon, fluids or gases.

Additional benefits and utilities of the present general inventive concept will be set forth in part in the description which follows and, in part, will be obvious from the description, or may be learned by practice of the general inventive concept

### SUMMARY OF THE INVENTIVE CONCEPT

The present inventive concept relates to a novel trans-cervical uterine entry device referred to as a cervical introducer, which can comprise a hollow threaded dilator body. The threaded dilator body portion of the cervical introducer is hollow to enable passage of instruments, such as, for example, an intrauterine device (IUD) inserter therethrough. The threaded dilator body portion features external threads for self-advancement through a cervix. The threaded dilator body portion preferably also includes slits along at least a portion of the length thereof originating from the distal tip to allow widening and accommodation of instruments to pass through. The cervical stoppage base is disposed at the proximal end and can include internal threads to move along the threaded dilator body portion and is generally in the form of a flat base to rest upon the cervix and prevent slippage of the trans-cervical uterine entry device during advancement of instruments therethrough.

The foregoing and/or other features and utilities of the present general inventive concept may be achieved by providing an atraumatic cervical introducer to enable non-traumatic insertion of medical devices into a cervical canal, comprising: an elongated tapered dilator body having an aperture extending entirely therethrough, external threads extending along a length of the outer surface of the dilator body and slits extending through the dilator body and threads from a first narrow end of the dilator body toward a second wider end thereof.

In an example embodiment, the dilator body is formed from at least one of polyethylene, polypropylene, polycarbonate, silicone, and stainless steel material.

In another example embodiment, the atraumatic cervical introducer can further include a cone shaped base attached at the second end of the dilator body.

In still another example embodiment, the dilator body and cone shaped base are formed from at least one of polyethylene, polypropylene, polycarbonate, silicone, and stainless steel material.

The foregoing and/or other features and utilities of the present general inventive concept may also be achieved by providing an atraumatic cervical introducer to enable non-traumatic insertion of medical devices into a cervical canal, comprising: an elongated dilator body having an aperture extending entirely therethrough and external threads extending along a length of the outer surface of the dilator body.

In an example embodiment, the dilator body is formed from at least one of polyethylene, polypropylene, polycarbonate, silicone, and stainless steel material.

The foregoing and/or other features and utilities of the present general inventive concept may also be achieved by providing an atraumatic cervical introducer to enable non-traumatic insertion of medical devices into a cervical canal, comprising: an elongated body having an aperture extending entirely therethrough and external threads extending along a length of the outer surface of the dilator body.

In an example embodiment, the dilator body is formed from at least one of polyethylene, polypropylene, polycarbonate, silicone, and stainless steel material.

Additional features and utilities of the present general inventive concept will be set forth in part in the description which follows and, in part, will be obvious from the description, or may be learned by practice of the general inventive concept.

### BRIEF DESCRIPTION OF THE DRAWINGS

These and/or other features and utilities of the present inventive concept will become apparent and more readily appreciated from the following description of the embodiments, taken in conjunction with the accompanying drawings of which:

FIG. 1 illustrates a conventional intrauterine device (IUD) with corresponding inserter used for cervical procedures.

FIG. 2 illustrates a prospective view of an atraumatic cervical introducer according to an example embodiment of the present inventive concept.

FIG. 2A illustrates a side view of the atraumatic cervical introducer according to the example embodiment of FIG. 2.

FIG. 2B illustrates a bottom lengthwise view of the atraumatic cervical introducer according to the example embodiment of FIG. 2.

FIG. 2C illustrates an operational side view of the atraumatic cervical introducer according to the example embodiment of FIG. 2.

FIG. 3 illustrates a prospective view of an atraumatic cervical introducer according to another example embodiment of the present inventive concept.

FIG. 3A illustrates a side view of the atraumatic cervical introducer according to the example embodiment of FIG. 3.

FIG. 4 illustrates a prospective view of an atraumatic cervical introducer according to another example embodiment of the present inventive concept.

FIG. 4A illustrates a side view of the atraumatic cervical introducer according to the example embodiment of FIG. 4.

The drawings illustrate a few example embodiments of the present inventive concept and are not to be considered limiting in its scope, as the overall inventive concept may admit to other equally effective embodiments.

### DETAILED DESCRIPTION OF THE PREFERRED EMBODIMENTS

Reference will now be made in detail to the embodiments of the present general inventive concept, examples of which are illustrated in the accompanying drawings, wherein like reference numerals refer to the like elements throughout. The embodiments are described below in order to explain the present inventive concept while referring to the figures. Also, while describing the present inventive concept, detailed descriptions about related well-known functions or configurations that may diminish the clarity of the points of the present general inventive concept are omitted.

It will be understood that although the terms “first” and “second” are used herein to describe various elements, these elements should not be limited by these terms. These terms are only used to distinguish one element from another element. Thus, a first element could be termed a second element, and similarly, a second element may be termed a first element without departing from the teachings of this disclosure.

Expressions such as “at least one of,” when preceding a list of elements, modify the entire list of elements and do not modify the individual elements of the list.

All terms including descriptive or technical terms which are used herein should be construed as having meanings that are obvious to one of ordinary skill in the art. However, the terms may have different meanings according to the intention of the lexicographer, case precedents, or the appearance of new technologies. Also, some terms may be arbitrarily selected by the inventors, and in this case, the meaning of the selected terms will be described in detail in the detailed description herein. Thus, the terms used herein should be defined based on the generally defined meaning of the terms together with the description throughout this specification.

New methods of trans-cervical uterine entry are discussed herein. In the following description, for purposes of explanation, numerous specific details are set forth in order to provide a thorough understanding of the present invention. It will be evident, however, to one skilled in the art that the present invention may be practiced without these specific details.

Hereinafter, one or more exemplary embodiments of the present general inventive concept will be described in detail with reference to accompanying drawings.

Example embodiments of the present general inventive concept are directed to an atraumatic cervical introducer, and more specifically, to an atraumatic cervical introducer for gynecologic procedures, which provides a hollow self-advancing threaded cervical dilator that enables passage of medical instruments.

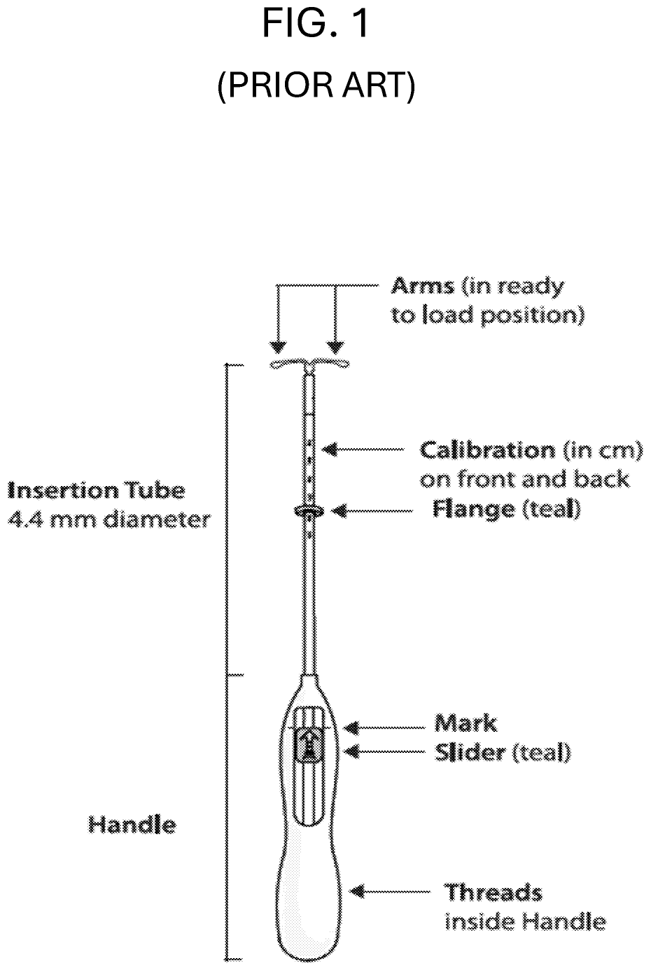

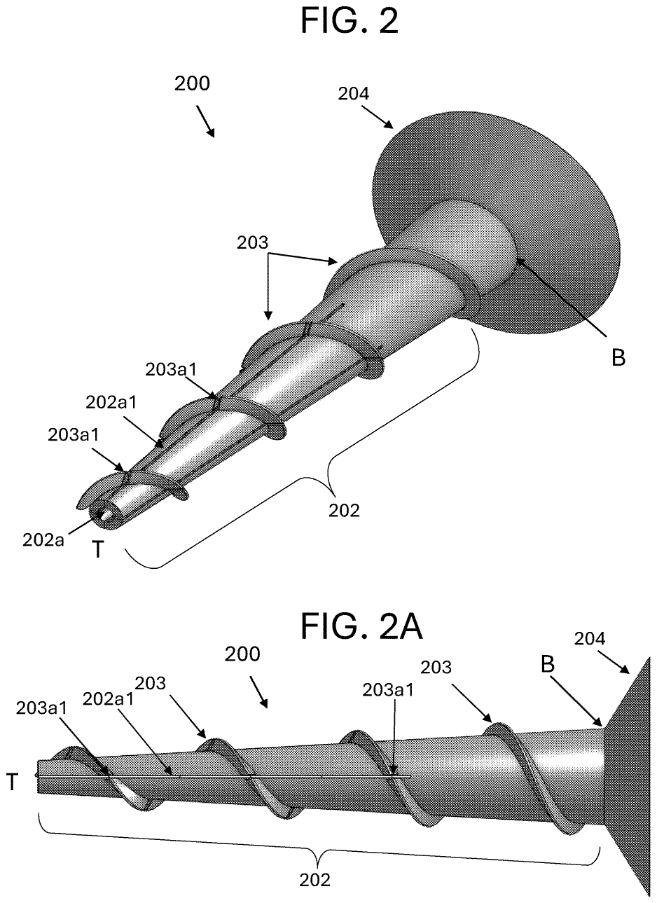

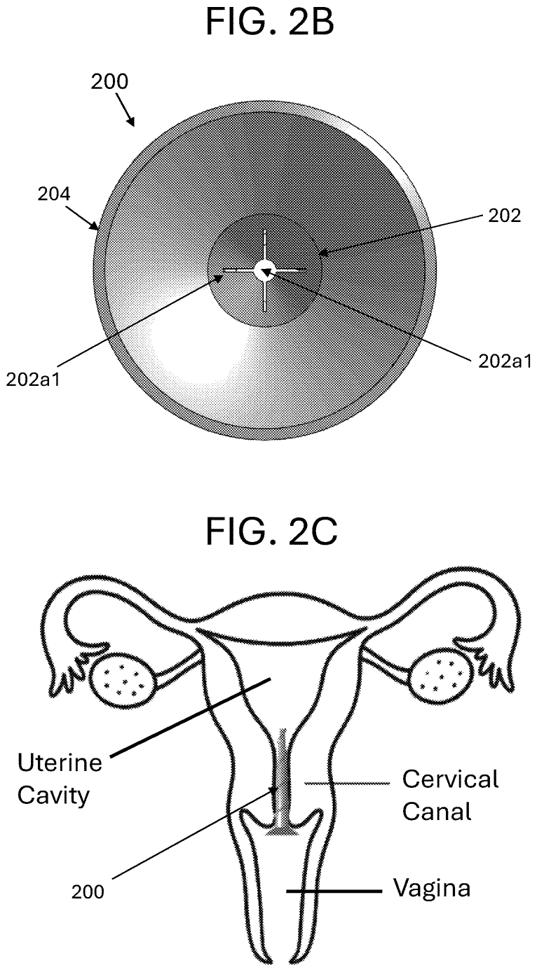

FIG. 2 illustrates a prospective view of an atraumatic cervical introducer 200, according to an example embodiment of the present inventive concept. FIG. 2A illustrates a side view of the atraumatic cervical introducer 200, according to the example embodiment of FIG. 2. The atraumatic cervical introducer 200 is designed to enable the insertion of an IUD and corresponding insertion tube therethrough, such as the IUD and insertion tube as illustrated in FIG. 1, or other medical devices.

Referring to FIGS. 2 and 2A, the atraumatic cervical introducer 200 can include an elongated hollow dilator body 202, which is preferably configured to be tapered in shape, increasing from a tip T (first end) thereof to a bottom B (second end) thereof. The hollow dilator body 202 preferably includes an aperture 202a extending entirely therethrough and external threads 203 extending along the length of the outer surface of the dilator body 202 from the tip T to the bottom B thereof. The aperture 202a is configured to be formed to a sufficient diameter to enable the insertion of an IUD and corresponding insertion tube therethrough, such as the IUD and insertion tube (see FIG. 1), or other medical devices.

The threads 203 formed on the outer surface of the dilator body 202 of the atraumatic cervical introducer 200 can be defined as helical ridges. An example embodiment may alternatively feature ridges (not illustrated), which are helical cutouts of the dilator body 202. The threads 203 are provided to convert gentle torque/rotation of the cervical introducer 200 to axial advancement through the cervical canal while creating a normal force to provide structural stabilization of the cervical os. The threads 203 are designed to reduce or eliminate cervical trauma when inserting the atraumatic cervical introducer 200 into the cervix and can be rounded at the edges thereof. Both the dilator body 202 and the threads 103 can be interrupted by open slits 202a1 and 203a1, respectively, which begin at the tip or first end of the dilator body 202 to allow the dilator body 202 (and therefore the aperture 202a) and the threads 203 to expand and accommodate instruments that are being passed within and through the atraumatic cervical introducer 200. For example, the slits 202a1 will create expanding legs which expand to increase the diameter of the aperture 202a. The atraumatic cervical introducer 200 can be constructed from medical-grade, sterile, flexible plastic materials, including, for example, polyethylene, polypropylene, and polycarbonate, to provide sufficient rigidity to guide medical devices and sufficient flexibility to prevent cervix and uterine injury. The atraumatic cervical introducer 200 may be alternatively made of medical grade silicone or medical grade stainless steel.

At the bottom (second end) of the dilator body 202 can be disposed a cervical stoppage base 204 which is provided to rest on the cervix and prevent slippage of the trans-cervical uterine entry device 200 during advancement of instruments, such as an IUD and inserter, therethrough. The stoppage base 204 is preferably, but not limited to, a cone shape.

FIG. 2B illustrates a bottom view of the atraumatic cervical introducer 200 according to the example embodiment of FIG. 2. As illustrated, the cervical stopper base 204 surrounds the bottom (second end) of the dilator body 202. Also illustrated is the aperture 202a and slits 202a1.

In an example embodiment the portion of the atraumatic cervical introducer 200 that is intended to go into the cervix is approximately 3-5 cm in length. The widest diameter of the portion of the atraumatic cervical introducer 200 that is intended to go into the cervix can be approximately 4 mm to 7 mm. The distal tip of the cervical introducer can be approximately 1-2 mm.

FIG. 2C illustrates an operational side view of the atraumatic cervical introducer according to the example embodiment of FIG. 2. Referring to FIG. 2C, the atraumatic cervical introducer 200 is illustrated as being positioned in the correct location in a cervical canal of a human's body. A clinician can position the cervical introducer 200 by inserting the front tip of the cervical introducer 200 at the external os of the cervix and advance the device through the cervical canal by gentle rotation of the device. Once the cervical introducer 200 is properly positioned the cervical introducer 200 will enable access to the uterine cavity from the vagina.

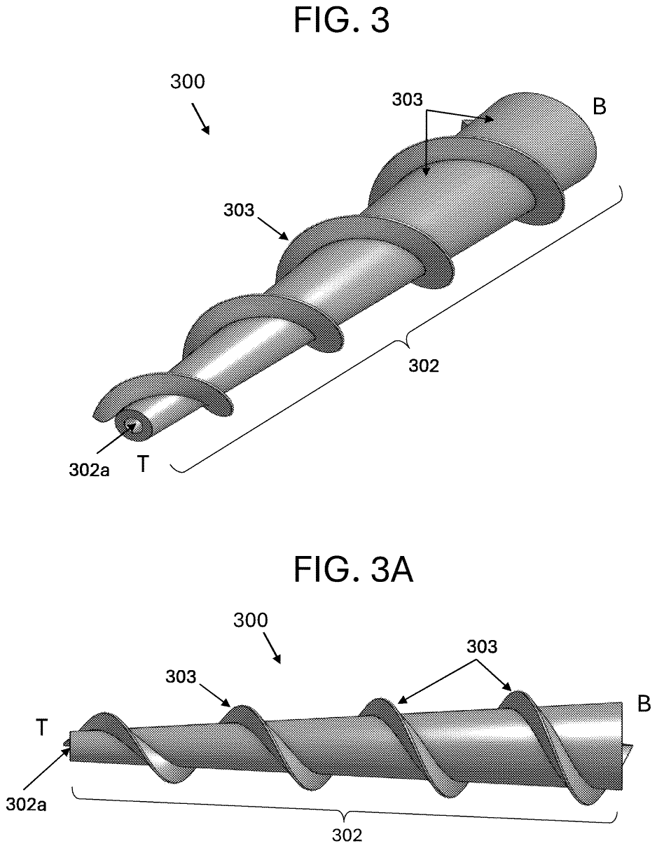

FIG. 3 illustrates a prospective view of an atraumatic cervical introducer 300 according to another example embodiment of the present inventive concept. FIG. 3A illustrates a side view of the atraumatic cervical introducer 300 according to the example embodiment of FIG. 3. The atraumatic cervical introducer 300 is designed to enable the insertion of an IUD and corresponding insertion tube therethrough, such as the IUD and insertion tube as illustrated in FIG. 1, or other medical devices.

Referring to FIGS. 3 and 3A, the atraumatic cervical introducer 300 can include an elongated hollow dilator body 302, which is preferably configured to be tapered in shape, increasing from a tip T (first end) thereof to a bottom B (second end) thereof. The hollow dilator body 302 preferably includes an aperture 302a extending entirely therethrough and external threads 303 extending along the length of the outer surface of the dilator body 302 from the tip T (first end) to the bottom B (second end) thereof. The aperture 302a is configured to be formed to a sufficient diameter to enable the insertion of an IUD and corresponding insertion tube therethrough, such as the IUD and insertion tube, or other medical devices. The atraumatic cervical introducer 300 can be constructed from medical-grade, sterile, flexible plastic materials, including, for example, polyethylene, polypropylene, and polycarbonate, to provide sufficient rigidity to guide medical devices and sufficient flexibility to prevent cervix and uterine injury. The atraumatic cervical introducer 200 may be alternatively made of medical grade silicone or medical grade stainless steel.

The threads 303 can be defined as helical ridges on the surface of dilator body 302. An example embodiment may alternatively feature ridges (not illustrated), which are helical cutouts of the dilator body 302. The threads 303 are provided to reduce or eliminate cervical trauma when inserting the atraumatic cervical introducer 300 into the cervix and can be rounded at the edges thereof.

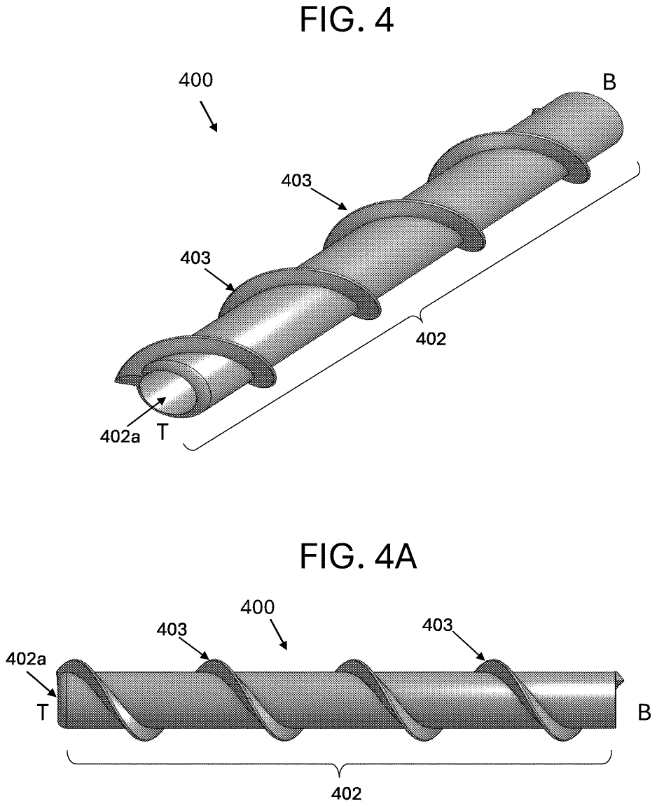

FIG. 4 illustrates a prospective view of an atraumatic cervical introducer 400 according to yet another example embodiment of the present inventive concept. FIG. 4A illustrates a side view of the atraumatic cervical introducer 400 according to the example embodiment of FIG. 4. The atraumatic cervical introducer 400 is designed to enable the insertion of an IUD and corresponding insertion tube therethrough, such as the IUD and insertion tube as illustrated in FIG. 1, or other medical devices.

Referring to FIGS. 4 and 4A, the atraumatic cervical introducer 400 can include an elongated hollow body 402. The hollow body 402 preferably includes an aperture 402a extending therethrough and external threads 403 extending along the length of the outer surface of the body 402 from a tip T (first end) to a bottom B (second end) thereof. The aperture 402a is configured to be formed to a sufficient diameter to enable the insertion of an IUD and corresponding insertion tube therethrough, such as the IUD and insertion tube, or other medical devices. The atraumatic cervical introducer 400 can be constructed from medical-grade, sterile, flexible plastic materials, including, for example, polyethylene, polypropylene, and polycarbonate, to provide sufficient rigidity to guide medical devices and sufficient flexibility to prevent cervix and uterine injury.

The threads 403 can be defined as helical ridges on the surface of body 402. An example embodiment may alternatively feature ridges (not illustrated), which are helical cutouts of the body 402. The threads 403 are provided to reduce or eliminate cervical trauma when inserting the atraumatic cervical introducer 400 into the cervix and can be rounded at the edges thereof.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. An atraumatic cervical introducer to enable non-traumatic insertion of medical devices through a cervical canal, comprising:
an elongated tapered dilator body having an aperture extending entirely therethrough, external threads extending along a length of the outer surface of the dilator body and slits extending through the dilator body and threads from a first narrow end of the dilator body toward a second wider end thereof.

2. The atraumatic cervical introducer according to claim 1, wherein the dilator body is formed from at least one of polyethylene, polypropylene, polycarbonate, silicone, and stainless steel material.

3. The atraumatic cervical introducer according to claim 1, further comprising:
a cone shaped base attached at the second end of the dilator body.

4. The atraumatic cervical introducer according to claim 3, wherein the dilator body and cone shaped base are formed from at least one of polyethylene, polypropylene, polycarbonate, silicone, and stainless steel material.

5. An atraumatic cervical introducer to enable non-traumatic insertion of medical devices into a cervical canal, comprising:
an elongated dilator body having an aperture extending entirely therethrough and external threads extending along a length of the outer surface of the dilator body.

6. The atraumatic cervical introducer according to claim 5, wherein the dilator body is formed from at least one of polyethylene, polypropylene, polycarbonate, silicone, and stainless steel material.

7. An atraumatic cervical introducer to enable non-traumatic insertion of medical devices into a cervical canal, comprising:
an elongated body having an aperture extending entirely therethrough and external threads extending along a length of the outer surface of the body.

8. The atraumatic cervical introducer according to claim 7, wherein the elongated body is formed from at least one of polyethylene, polypropylene, polycarbonate, silicone, and stainless steel material.
